(19) United States
(12) Patent Application Publication
(10) Pub. No.: US 2025/0325455 A1
(43) Pub. Date: Oct. 23, 2025

(54) SOLID COSMETIC MAKE-UP COMPOSITION COMPRISING AT LEAST ONE CAVITY

(71) Applicant: CAPSUM, MARSEILLE (FR)

(72) Inventors: Sébastien BARDON, VEYRIER DU LAC (FR); Mathieu GOUTAYER, SAINT MALO (FR); Jean-François GONIDEC, AIX-EN-PROVENCE (FR)

(21) Appl. No.: 18/859,467

(22) PCT Filed: Apr. 26, 2023
(86) PCT No.: PCT/EP2023/060973

§ 371 Date: Oct. 23, 2024

(30) Foreign Application Priority Data
Apr. 26, 2022 (FR) FR2203883

(51) Int. Cl. A61K 8/02; A61Q 1/06

(52) U.S. Cl. CPC A61K 8/0216; A61Q 1/06; A61K 2800/88

(57) ABSTRACT

The present disclosure relates to a solid cosmetic make-up composition of a keratin material, in particular the skin and/or the lips, comprising at least one pigment, and at least one cavity such that the composition has a porosity of greater than or equal to 20% by volume relative to the total volume of the solid composition, as well as to associated production methods.

The invention relates to a solid cosmetic composition intended for the make-up application to a keratin material, more particularly the skin and/or the lips.

Solid cosmetic compositions intended for the make-up application to a keratin material in the form of sticks (i.e. lipstick, foundation, blush), hot casts or compacted powders are already known.

However, such solid make-up compositions, generally anhydrous, do not give total satisfaction in terms of homogeneity of deposition, of comfort, of sensoriality and/or of hydration. Indeed, the depositions are generally greasy and/or sticky.

Moreover, with regard to sticks, the use thereof involves repeated contact between the solid composition and the skin and/or the lips which, for obvious reasons, is not very hygienic. What is more, the degradation of the sticks and/or the rupture thereof before the total consumption there generates wastage and hence waste.

With regard to hot castings, the conditioning thereof often involves a lot of packaging which is not desirable from an ecological point of view. More particularly, hot castings generally rely on indirect application via an application device, e.g. a brush, which requires post-use cleaning, which is restrictive and not very hygienic.

Solid cosmetic compositions intended make-up in the form of single doses, are already known. However, the compositions comprise a lot of packaging, and/or are endowed with properties that are not entirely satisfactory in terms of gripping, ease of application, sensoriality, hydration and/or good feeling during application.

The present invention aims to overcome the aforementioned drawbacks by proposing solid cosmetic make-up compositions, more particularly in the form of a single dose, guaranteeing good grip by the user, direct and comfortable application, and economical packaging.

### Composition

The invention thereby relates to a solid cosmetic make-up composition of a keratin material, more particularly for the skin and/or the lips, comprising at least one pigment, characterized in that the said composition comprises at least one cavity such that said composition comprises a porosity greater than or equal to 20%, preferably greater than or equal to 30%, better greater than or equal to 40%, more particularly greater than or equal to 50%, more particularly greater than or equal to 70%, and most particularly greater than or equal to 80%, by volume relative to the total volume of said solid composition.

Thereby, a composition according to the invention may remain endowed with a size and/or a volume similar to current solid make-up compositions but differs in the presence of at least one cavity such that the porosity of said composition is greater than or equal to 20% by volume relative to the total volume of said composition.

“Solid”, as defined by the present invention, refers to a composition which, at ambient temperature and at atmospheric pressure, is not apt to collapse under its own weight. Advantageously, one intends to refer to any composition having a compressive strength of greater than or equal to 20 g, preferably greater than or equal to 50 g, and better greater than or equal to 100 g, at ambient temperature (20-25° C.), after penetration by a cylindrical probe of revolution having a diameter of 0.8 cm into the matrix of the composition in a thickness of 1 mm at a speed of 0.5 mm/s, and removal of said probe from the matrix of the composition at a speed of 0.5 mm/s; the compression strength being measured with an analyzer such as a “LFRA Texture Analyzer” marketed by STEVENS/MECHTRIC.

A composition according to the invention is not in the form of a paste. A composition according to the invention is not a pulverulent composition, and hence is not in the form of a powder. Finally, a composition according to the invention is neither a porous solid derived from the agglomeration of powders. On the other hand, a composition according to the invention may be a puffed composition, and hence may be in the form of a foam. According to a particular embodiment, a composition according to the invention is not a puffed composition, and hence is not in the form of a foam.

Preferably, a solid composition according to the invention is not coated with a water-soluble coating.

“Porosity”, as defined by the present invention, refers to all the voids of a solid material, the voids being able to be filled with fluids, in particular water, when the solid composition is brought into contact with an aqueous solution, e.g. water such as tap water.

“Cavity”, as defined by the present invention, refers to an empty space inside a solid body and which communicates directly with the external medium, like e.g. the cavities illustrated in FIGS. 1 to 3 and 6. A cavity can thus be referred to, without distinction, by the terms “orifice”, “pore” or “hole”. Advantageously, the cavity (ies) is/are through, i.e. the cavity designates an empty space inside a solid body and which communicates directly with the external medium on both sides of the solid body.

Thereby, the volume of the cavity (ies) in a solid composition according to the invention represents at least 20%, more particularly at least 30%, preferably at least 40%, more particularly at least 50%, even at least 60%, more particularly at least 70%, and most particularly at least 80%, of the total volume of said solid composition.

Advantageously, a solid composition according to the invention has a density comprised between 0.2 and 0.8, preferably between 20 and 0.7, more particularly between 20 and 0.6.

A solid composition according to the invention may have a total volume comprised between 0.125 cm³ and 100 cm³, preferably between 0.250 cm³ and 75 cm³, more particularly between 0.5 cm³ and 50 cm³, better between 1 cm³ and 25 cm³, and most particularly between 2.5 cm³ and 15 cm³.

A solid composition according to the invention may have a weight comprised between 0.025 g and 200 g, preferably between 0.25 g and 150 g, more particularly between 0.5 g and 100 g, better between 1 g and 75 g, most particularly between 2.5 g and 50 g, even between 5 g and 25 g (“g” corresponding to the “gram” unit).

Advantageously, a solid composition according to the invention may have a weight comprised between 0.25 g and 200 g, preferably between 0.50 g and 150 g, more particularly between 1 g and 100 g, better between 2.5 g and 75 g, and most particularly between 5 g and 50 g (“g” corresponding to the “gram” unit).

According to a first preferred embodiment, a solid composition according to the invention comprises a ratio between the volume of the cavities and the total volume of the composition comprised between 0.05 and 0.9, preferably between 0.1 and 0.8, more particularly between 0.2 and 0.9, preferably between 0.2 and 0.8, better between 0.2 and 0.7, even between 0.2 and 0.6.

“Total volume” refers to the volume resulting from the sum of the volume of solid and the of the volume of the cavities.

Within the framework of the present invention, the cavities may be considered to be macroporosity, i.e. pores the minimum width, the diameter even, of which is greater than or equal to 0.5 mm, preferably greater than or equal to 0.75 mm, and more particularly greater than or equal to 1 mm, even comprised between 0.5 mm et 2 cm, most particularly between 1 mm et 2 cm, preferably between 2 mm et 1 cm, and more particularly between 3 mm et 75 mm, better between 4 mm et 50 mm, and most particularly between 5 mm et 25 mm. More particularly, the pores have a minimum width, even a diameter, comprised between 0.5 mm et 5 mm, preferably between 1 mm and 3 mm.

As such, the Applicant indicates that, at equivalent porosity, manufacturing a stable solid composition is:
easy in the presence of small cavities, e.g. the cavities present in solid substrates where the porosity is consecutive to the addition of a gas during the manufacturing process, as described in U.S. Pat. No. 8,349,341, but
much more difficult in the presence of macroporosities such as same considered in the present invention.

The presence of cavities in the contents and/or volumes and/or dimensions considered hereinabove makes it possible to obtain solid compositions which require less raw materials while remaining endowed with satisfactory gripping properties. A solid composition according to the invention is thus apt to be put in a single-dose format generating no loss or less loss. Such weight saving further has the advantage of permitting less polluting and less expensive transport. A solid composition according to the invention is thus both ecologically and economically advantageous.

Moreover, the inventors observed an unexpected effect in terms of texture and transformative effect when a solid composition according to the invention is placed in the presence of an aqueous phase. Indeed, the inventors have observed that a solid composition according to the invention, in contact with an aqueous phase, exhibits a faster transformation kinetics between the original solid state and a liquid state, e.g. such as a gel or a cream, compared to a solid composition of the same size and volume, but without any cavity. Without wishing to be bound by any theory, the inventors believe that the presence of cavity (ies) within the solid composition makes it possible to facilitate the transition to a viscous liquid composition, e.g. when the solid composition is mixed with the hands, or leads to an increase in the contact surface area with the aqueous phase, more particularly water, and thus a better hydration of said solid composition.

Thereby, the invention even makes it possible to regulate the speed at which the solid composition will swell and/or melt, by adjusting the developed surface to constant volume, by regulating the percentage of porosity, and more particularly by acting on the shape and/or the number and/or dimensions of the cavities, even the number of walls and/or the thickness of the walls of the cavities.

Advantageously, the size and/or the volume of the cavities can be adjusted to store and/or react with the right amount of water necessary to achieve a final liquid composition endowed with satisfactory/expected properties, even optimized and/or homogeneous (i.e. between different single doses), e.g. in terms of viscosity, hydration, ease of spreading and/or transformative effect.

Thereby, a composition according to the invention even makes it possible to obtain evanescent compositions, namely compositions apt to be transformed into liquid compositions, more particularly in the form of gels or creams, in contact with an adequate quantity of water.

Finally, the inventors have observed that the porous structure of a solid composition according to the invention makes it possible to obtain depositions on keratin materials which are more homogeneous, even without recourse to application members.

The solid character of a composition according to the invention may be based on a phase consisting predominantly, or even totally, of a fatty phase comprising at least one lipophilic gelling agent.

Preferably, the solid character of a composition according to the invention is based on the presence of a fatty phase comprising at least one lipophilic gelling agent.

According to a particular embodiment, a composition according to the invention may also comprise an aqueous phase dispersed in the solid composition, preferably in encapsulated form.

Preferably, a composition according to the invention is a single-dose composition. Such embodiment is particularly advantageous from a hygienic point of view in that same prevents repeated contacts between the solid composition and the keratin material. Such embodiment also makes it easy for the user to adapt the desired shade according to his/her state of mind (mood), his/her style of clothing and/or the time of day.

Thereby, the invention further relates to a packaging comprising a plurality of solid compositions according to the invention in the form of single-doses with different shades. Thereby, the invention relates to a packaging comprising a plurality of solid compositions according to the invention, preferably in the form of single doses, said compositions comprising different pigments and/or different contents of pigments and therefore different shades. Such packaging thus has the advantage of providing an easy customization of the shade sought by the user according to his/her state of mind (mood), his/her style of clothing and/or the time of day.

Preferably, a composition according to the invention is a topical composition. A composition according to the invention is not an oral composition.

A solid composition according to the invention may have any shape, e.g. a spherical, ellipsoid, tetrahedral or polygonal shape, more particularly in the shape of a parallelogram, preferably a rectangular parallelogram.

Preferably, in particular to provide a good grip, a solid composition according to the invention has:
a width, or even a diameter, comprised between 1 cm and 10 cm, preferably between 2 cm and 8 cm, more particularly between 3 cm and 6 cm, and better between 4 cm and 5 cm; and/or
a height (or thickness) of between 0.5 cm and 5 cm, preferably between 1 cm and 4 cm, and more particularly between 2 cm and 3 cm.

The cavity (ies) of a solid composition according to the invention can have any shape, e.g. a round, oblong, ovoid, triangular, polygonal, more particularly hexagonal, honeycomb shape, or in the shape of a paving (or cutout), e.g. of such as a Voronoï diagram, preferably in the shape of a honeycomb, as illustrated in FIGS. 3 and 6.

The cavity (ies) of a solid composition according to the invention can also represent letters or the logo of a brand or a company, as illustrated in FIG. 2.

A solid composition according to the invention comprises one or a plurality of cavities, preferably a plurality of cavities, either identical or different.

According to a first variant, the composition according to the invention comprises at least one cavity, more particularly a through cavity.

According to a second preferred variant, a composition according to the invention comprises a plurality of cavities, more particularly through cavities, as illustrated in FIGS. 3 and 6.

The cavities are through cavities, advantageously along an axis x-x′.

According to a particular embodiment, a composition according to the invention may comprise at least two cavities connected together. Such an embodiment is advantageous because same enhances capillarity phenomena when brought into contact with water, which improves the aforementioned technical effects.

Advantageously, a solid composition according to the invention comprises a plurality of cavities which take a honeycomb-shaped structure, as illustrated in FIG. 3. The honeycomb shape is particularly advantageous in that same permits the manufacture of a solid composition provided with a very advantageous “volume of the cavity (ies)/total volume of the composition” ratio, more particularly greater than or equal to 0.5, even greater than or equal to 0.75, without prejudice to the grip and the robustness of said composition to the mechanical stresses likely to be applied thereto and/or to storage. In other words, the honeycomb structure is an excellent compromise between cavity volume and mechanical strength.

Advantageously, the cavity (ies) of a solid composition according to the invention comprise(s) walls having a thickness comprised between 10 μm and 1,500 μm, preferably between 20 μm and 1,000 μm, more particularly between 30 μm and 750 μm, most particularly between 50 μm and 500 μm, even between 100 μm and 400 μm. More particularly, the cavity (ies) of a solid composition according to the invention comprises/comprise walls having a thickness comprised between 250 μm and 500 μm.

Advantageously, a solid composition according to the invention comprises a plurality of cavities which take a honeycomb-shaped structure comprising walls having a thickness comprised between 10 μm and 600 μm, preferably between 20 μm and 500 μm, more particularly between 30 μm and 400 μm, most particularly between 50 μm and 300 μm, even between 100 μm and 200 μm.

According to a particular embodiment, a solid composition according to the invention, in particular when same takes a honeycomb configuration as described hereinabove, may comprise a plurality of sections, more particularly polygonal, each of the sections defining a breakable perimeter permitting the separation of at least one section from the remainder of the solid composition. Thereby, according to such embodiment, each section of the solid composition corresponds to a single dose.

According to a particular embodiment, a composition according to the invention may also comprise additional patterns distinct from the cavities, said patterns being able, e.g., to be produced by embossing.

According to a first variant, a composition according to the invention is anhydrous. “Anhydrous”, as defined by the present invention, refers to a composition which, before any contact with water, comprises a water content of less than 5% by weight, preferably less than 1% by weight and more particularly less than 0.5% by weight, relative to the total weight of said composition.

According to a second variant, a composition according to the invention comprises a continuous fatty phase and a dispersed aqueous phase. Thereby, a composition according to the invention is in the form of an oily (or fatty) phase which additionally comprises at least one dispersed aqueous phase, e.g. in encapsulated form such as e.g. in the form of matrix spheres (or beads or small balls) or such as cores/shells (or capsules) e.g. in the form of capsules as described in WO2010/063937.

Thereby, a solid composition according to the invention may also comprise at least one aqueous phase dispersed in the solid composition, preferably in encapsulated form.

According to one embodiment, the mass percentage of water of the aqueous phase may then be comprised between 0.5% and 50%, preferably between 1%, and 40%, more particularly between 2.5% and 30%, and better between 5% and 20%, by weight relative to the total weight of the composition.

Preferably, the solid character of a composition according to the invention is based on the presence of a fatty phase comprising at least one lipophilic gelling agent.

According to a first variant, a composition according to the invention is anhydrous. “Anhydrous”, as defined by the present invention, refers to a composition which, before any contact with water, comprises a water content of less than 5% by weight, preferably less than 1% by weight and more particularly less than 0.5% by weight, relative to the total weight of said composition.

According to a second variant, a composition according to the invention comprises a continuous fatty phase and a dispersed aqueous phase. Thereby, a composition according to the invention is in the form of an oily (or fatty) phase which additionally comprises at least one dispersed aqueous phase, e.g. in encapsulated form such as e.g. in the form of matrix spheres (or beads or small balls) or such as cores/shells (or capsules) e.g. in the form of capsules as described in WO2010/063937.

Thereby, a solid composition according to the invention may also comprise at least one aqueous phase dispersed in the solid composition, preferably in encapsulated form.

According to one embodiment, the mass percentage of water of the aqueous phase may then be comprised between 0.5% and 50%, preferably between 1%, and 40%, more particularly between 2.5% and 30%, and better between 5% and 20%, by weight relative to the total weight of the composition.

### Lipophilic Gelling Agent

A composition according to the invention advantageously comprises at least one lipophilic gelling agent.

The lipophilic gelling agent according to the invention may be chosen from organic or inorganic, polymeric or molecular lipophilic gelling agents; fats which are solid at ambient temperature and pressure; and mixtures thereof.

The fatty substances which are solid at ambient temperature and pressure are preferably chosen from at least one wax, at least one pasty fatty substance, at least one butter, and mixtures thereof.

Advantageously, a lipophilic gelling agent is a heat-sensitive gelling agent, namely one which reacts to heat, and in particular is a gelling agent which is solid at ambient temperature and liquid at a temperature greater than 50° C., preferably greater than 60° C., and better greater than 70° C. Preferably, a heat-sensitive lipophilic gelling agent according to the invention has a melting point comprised between 50° C. and 130° C., and preferably between 60° C. and 120° C.

Lipophilic gelling agent(s) include e.g. of same described in WO2021/234135.

According to a particularly preferred embodiment, the lipophilic gelling agent is chosen from Castor Oil/IPDI copolymer (and) Caprylic/Capric Triglyceride, marketed in particular under the name Estogel M by PolymerExpert, Hydrogenated Castor Oil/Sebacic Acid Copolymer and mixtures thereof, in particular marketed under the names Estogel Green (or Estogel G) and Estogel Green 40, respectively, by PolymerExpert, Caprylic/Capric Triglyceride (and) Polyurethane-79, marketed in particular under the name OILKEMIA™ 5S polymer by the company Lubrizol, Trihydroxystearin, in particular marketed under the name THIXCIN® R by the company Elementis Specialties, and mixtures thereof, and, better, Castor Oil/IPDI Copolymer (and) Caprylic/Capric Triglyceride.

More particularly, a composition according to the invention may comprise from 10% to 90%, preferably from 20% to 80%, more particularly from 30% to 70%, and better from 40% to 60%, by weight of lipophilic gelling agent(s) relative to the total weight of the composition.

### Oils

A composition according to the invention can comprise at least one oil. “Oil” refers to a fat that is liquid at ambient temperature and atmospheric pressure.

As oils according to the invention, mention may be made e.g. of:
hydrocarbon oils of vegetable origin, as described hereinafter;
hydrocarbon oils of animal origin, such as perhydrosqualene and squalane;
synthetic esters and ethers, in particular of fatty acids, such as oils with the formulae R1COOR2 and R1OR2 wherein R1 represents the residue of a C₈ to C₂₉ fatty acid and R2 represents a C₃ to C₃₀ hydrocarbon chain, branched or unbranched, such as Purcellin oil, isononyl isononanoate, isodecyl neopentanoate, isostearyl neopentanoate, isopropyl myristate, ethyl-2-hexyl palmitate, octyl-2-dodecyl stearate, octyl-2-dodecyl erucate, isostearyl isostearate; hydroxy esters such as isostearyl lactate, octylhydroxystearate, octyldodecyl hydroxystearate, diisostearyl malate, triisocetyl citrate, heptanoates, octanoates, decanoates of fatty alcohols; polyol esters such as propylene glycol dioctanoate, neopentyl glycol diheptanoate and diethylene glycol diisonanoate; and pentaerythritol esters such as pentaerythrityl tetrabehenate (DUB PTB) or pentaerythrityl tetraisostearate (Prisorine 3631);
linear or branched hydrocarbons of mineral or synthetic origin, such as paraffin oils, either volatile or non-volatile, and the derivatives thereof, petroleum jelly, polydecenes, hydrogenated polyisobutene such as Parleam oil;
silicone oils, such as volatile or non-volatile polymethylsiloxanes (PDMS) with a linear or cyclic silicone chain, liquid or pasty at room temperature, in particular cyclopolydimethylsiloxanes (cyclomethicones) such as cyclohexasiloxane and cyclopentasiloxane; polydimethylsiloxanes (or dimethicones) containing alkyl, alkoxy or phenyl groups, during or at the end of the silicone chain, groups containing from 2 to 24 carbon atoms; phenylsilicones such as phenyltrimethicones, phenyldimethicones, phenyltrimethylsiloxydiphenylsiloxanes, diphenyldimethicones and diphenylmethyldiphenyltrisiloxanes, 2-phenylethyltrimethyl siloxysilicates, and polymethylphenylsiloxanes;
fatty alcohols with 8 to 26 carbon atoms, such as cetyl alcohol, stearyl alcohol and the mixture (cetyl stearyl alcohol) thereof, or else octyldodecanol;
partially hydrocarbon-based and/or silicone-based fluorinated oils such as same described in document JP-A-2-295912;
and mixtures thereof.

As oils, mention may be made e.g. of same described in WO2021/234135.

A composition according to the invention preferably comprises at least one vegetable oil, such as e.g. copra oil, coconut oil, olive oil, sweet almond oil, castor oil, Babassu oil, and mixtures thereof.

More particularly, a composition according to the invention may comprise from 10% to 90%, preferably from 20% to 80%, more particularly from 30% to 70%, and better from 40% to 60%, by weight of oil(s) relative to the total weight of the composition.

A composition according to the invention may comprise at least one lipophilic gelling agent, preferably heat-sensitive, at least one oil and mixtures thereof.

### Pigments

A solid composition according to the invention comprises at least one pigment. “Pigment” refers to a coloring chemical substance insoluble in the phase wherein the pigment is present. “Insoluble” means that the solubility at 20° C. of the pigment in the phase wherein the pigment is present is less than 1 g/l, in particular less than 0.1 g/l, preferably less than 0.001 g/l.

Each pigment may independently be an organic, inorganic or organic-inorganic hybrid pigment. The pigments are typically inorganic pigments.

The coloration imparted by a composition according to the invention can e.g. be measured by spectrocolorimetry and/or spectrophotocolorimetry.

Coverage corresponds to the capacity of a composition to “mask the skin”/to “hide imperfections”. The coverage of a composition according to the invention can be measured according to the following method.

The composition is spread on matt black and matt white contrast cards, e.g. made by LENETA Form WPL for the matt black card and Leneta IA for the matt white card. The application can be done with an automatic spreader. The measurements are carried out on the compositions thereby spread. Reflectance spectra are acquired using a Minolta 3700-D spectrocolorimeter (diffuse measurement geometry and observation D65/10°, specular component mode excluded, small aperture (CREISS)) on black and white backgrounds. The spectra are expressed in colorimetric coordinates in the CIELab76 space within the meaning of the International Commission on Lighting according to the recommendation 15:2004. The contrast ratio, or coverage, is calculated by taking the arithmetic mean of Y on a black background divided by the mean value of Y on a white background, multiplied by 100.

Pigments include e.g. titanium dioxide, zinc dioxide, zirconium or cerium oxides, iron or chromium oxides, manganese violet, ultramarine blue, chromium hydrate and ferric blue, and mixtures thereof. Preferred inorganic pigments are iron oxides, in particular red iron oxide, yellow iron oxide, brown iron oxide, black iron oxide, titanium dioxide and mixtures thereof.

The pigment is preferably an iron oxide, especially red iron oxide, yellow iron oxide, brown iron oxide, black iron oxide and mixtures thereof.

Each pigment may be an untreated pigment or a treated pigment. “Treated pigment”, as defined by the application, refers to a pigment which has been treated with an additive improving the dispersibility thereof in an oily or aqueous composition, in particular one of the additives defined hereinbelow. “Untreated pigment” or “not treated pigment” refers to a pigment which has not been treated with such an additive.

According to a first alternative, the pigment used is an untreated and unground pigment (pigment used “as is”).

According to a second alternative, the pigment used has undergone a prior treatment in order to make same more easily dispersible during the formulation of the pigment, i.e. in particular more easily dispersible in the phase considered. Such pretreatment consists in grinding the pigment and/or pretreating same with an additive improving the dispersibility of the pigment before formulating same into a series of colored particles.

The use of a ground pigment and/or a pigment pretreated with an additive improving the dispersibility thereof:
contributes to the preparation of a composition the fatty phase of which has a high pigment content,
contributes to the reduction of, or even prevents, the sedimentation of the pigment(s) in the phase(s) containing same, and/or
contributes to the decrease of, or even prevents, the aggregation of pigments in the phase(s) containing same.

Generally, when a plurality of pigments are used, same all undergo the same treatment, i.e. same are all ground and/or pretreated. However, it is possible that certain pigments may be ground and untreated, and others treated and ground or unground.

According to a first embodiment according to the second alternative, the at least one pigment is pretreated with an additive improving the dispersibility of the pigment.

The nature of the additive improving the dispersibility of the pigment depends on the hydrophilic or lipophilic nature of the phase(s) which will comprise the treated pigment.

When a composition uses a plurality of pre-treated pigments, the pigments may be pre-treated with additives that are identical or different from each other.

An additive improving the dispersibility of the pigment within an oily phase is chosen e.g. from hydrogenated lecithin, a silicone, a wax, an amino acid or one of the salts thereof and an amino acid ester or one of the salts thereof, and mixtures thereof. Hydrogenated lecithin includes phosphate mono-and di-esters comprising fatty chains which enhance dispersibility in the oily phase. The silicone additive may either be obtained from a silicone precursor, such as an alkoxyalkylsilane such as triethoxycaprylsilane, or such as a trialkylsiloxysilicate such as trimethylsiloxysilicate, or be a silicone such as dimethicone or a derivative thereof, such as bis-hydroxyethoxypropyl dimethicone, or be obtained from a mixture of silicone and one of the precursors thereof, e.g. a mixture of dimethicone and trimethylsiloxysilicate. The silicone additive can be a hybrid treatment, more particularly a mixture of isopropyl titanium triisostearate, bis-hydroxyethoxypropyl dimethicone, PEG-2 soyamine and isophorone diisocyanate (IPDI). The wax may be e.g. rose floral wax. The preferred amino acid is cystine, and preferred amino acid esters are sodium cocoyl glutamate, layroyl arginine or lauroyl lysine.

As pigments, additives improving the dispersibility of pigments in the oily or aqueous phase, and method for preparing a pigment pretreated with an additive, mention may be made e.g. of same described in WO2018178353 or FR2112468.

The solid character of a composition according to the invention advantageously permits the presence of pigments in high concentrations, which makes it possible to reconcile in the same composition opposing technical performances such as coverage and homogeneity of the deposition, even comfort in application and good hydration.

A person skilled in the art would make sure to choose the pigments and/or the amount thereof so that the advantageous properties of the composition according to the invention, are not altered or not substantially altered by the envisaged admixture. Also, a person skilled in the art would make sure to choose the nature and/or the amount of pigments depending on the aqueous or fatty nature of the phase considered and/or with regard to the method for manufacturing the composition. Such adjustments fall within the general knowledge of a person skilled in the art.

More particularly, a composition according to the invention comprises between 1% and 60%, preferably between 5% and 50%, more particularly between 10% and 40%, more particularly better between 15% and 35%, and preferably between 20% and 25%, by weight of pigment(s) relative to the total weight of said composition.

### Additional Compounds

A composition according to the invention, more particularly the fatty phase, even the dispersed aqueous phase when present, may further comprise at least one additional compound different from the aforementioned lipophilic gelling agent, oils and pigments.

A composition according to the invention may also comprise powders; flakes; coloring agents, in particular chosen from water-soluble or water-insoluble, liposoluble or non-liposoluble, organic or inorganic coloring agents, materials with an optical effect, liquid crystals, and mixtures thereof; particulate agents insoluble in the fatty phase; scenting agents; preservatives; humectants; fillers with a blurring effect; effervescent agents; disintegrants (e.g. Croscarmellose, carboxymethylcellulose, etc.); thinners; disintegration-promoting agents; stabilizers; chelating agents; softeners; modifying agents chosen amongst texture agents (or gelling agents) and more particularly hydrophilic gelling agents, agents modifying the pH, the osmotic force and/or modifiers of the refractive index etc. . . . or any usual cosmetic additive; and mixtures thereof.

A composition according to the invention, more particularly the fatty phase, even the dispersed aqueous phase when present, may comprise at least one biological/cosmetic active agent, in particular chosen from moisturizing agents, anti-wrinkle or anti-aging agents, vitamins, cicatrizing agents, depigmenting agents, UV filters, desquamating agents, antioxidants, active agents stimulating the synthesis of dermal and/or epidermal macromolecules, skin relaxant agents, antiperspirants, soothing agents and/or anti-aging agents, and mixtures thereof.

As hydrophilic gelling agents, mention may be made of same described in FR3041251.

A solid composition according to the invention can further comprise at least one blur effect filler. A blur effect filler can modify and/or mask wrinkles by the intrinsic physical properties thereof. Such fillers may in particular modify wrinkles via a tensor effect, a camouflage effect or a blur effect.

Examples of the following compounds may be given as blur effect fillers:
silica porous microparticles e.g. Silica Beads® SB 150 and SB 700 by Miyoshi having a mean size of 5 μm, and SUNSPHERES® H series by Asahi Glass such as H33, H51, of size 3.5 and 5 μm respectively, and Sensibead Si 175 and Sensibead Si 320 by Sensient Cosmetic Technologies of size 7 μm and 5 μm, respectively;
hollow hemispherical particles of silicone resins such as NLK 500®, NLK 506® and NLK 510® from Takemoto Oil and Fat, described in particular in EP 1 579 849;
silicone resin powders such as SILICON Resin Tospearl® 145 A DE GE silicone of mean size 4.5 μm;
powders of acrylic copolymers in particular of methyl poly (meth) acrylate, e.g. the particles PMMA Jurimer MBIR by Nihon Junyoki of mean size 8 μm, hollow PMME spheres sold under the trade name COVABEAD® LH 85 by Sensient Cosmetic Technologies, and expanded microspheres of vinylidene/acrylonitrile/methylene methacrylates sold under the trade name Expancel®;
wax powders such as Paraffin wax microloase® 114S particles from Micropowders with a mean size of 7 μm;
polyethylene powders, in particular comprising at least one ethylene/acrylic acid copolymer such as, e.g., Sumimoto's FLOBEADS® EA 209 E with a mean size of 10 μm;
crosslinked elastomeric organopolysiloxane powders coated with silicone resin, in particular silsesquioxane, under the name KSP 100®, KSP 101®, KSP 102®, KSP 103®,KSP 104® and KSP 105® by the company Shin Etsu;
talc/dioxide or titanium/alumina/silica composite powders such as Coverleaf AR 80® from

### Catalyst & Chemical;

talc, mica, kaolin, lauryl glycine, starch powders crosslinked by octeanyl succinate anhydride, boron nitride, polytetrafluoroethylene powders, precipitated calcium carbonate, magnesium hydrocarbonate carbonate, barium sulfate, hydroxyapatite, calcium silicate, cerium dioxide and glass or ceramic microcapsules;
synthetic or natural, mineral or organic hydrophilic or hydroplethobic fibers such as silk, cotton, wool, flax, cellulose fibers extracted in particular from wood, vegetables or algae, polyamide (Nylon®), modified cellulose, poly-p-phenylene terephtamide or acrylic fibers, polyolefin, glass, silica, aramid, carbon, polytetrafluoroethylene (Teflon®), insoluble collagen, polyesters, polyvinyl chloride or vinylidene chloride, polyvinyl alcohol, polyacrylonitrile, chitosan, polyurethane, of polyethylene phthalate, fibers formed from a mixture of polymers, resorbable synthetic fibers, and mixtures thereof described in the patent application EP 1 151 742;
spherical elastomer crosslinked silicones such as Trefil E-505C® or E-506 C® from Dow Corning;
abrasive fillers which, by mechanical effect, provide a smoothing of the skin microrelief, such as abrasive silica such as e.g. Abrasif SPR from Semanez or powders of nuts or shells (apricots, nuts e.g. from Cosmetochem); and
mixtures thereof.

A composition according to the invention may also comprise at least one solvent apt to evaporate, completely or partially, during the manufacturing process, as described hereinafter. As solvent, mention may be made e.g., of an apolar or slightly polar organic solvent, e.g. cyclohexane, THF (tetrahydrofuran), and mixtures thereof.

Of course, a person skilled in the art would make sure to choose any additional compound(s) and/or the amount thereof so that the advantageous properties of the composition according to the invention, are not altered or not substantially altered by the envisaged admixture. Also, a person skilled in the art would make sure to choose the nature and/or the amount of additional compound(s) depending on the aqueous or fatty nature of the phase considered and/or with regard to the method of manufacturing the composition. Such adjustments fall within the general knowledge of a person skilled in the art.

The solid character of a composition according to the invention advantageously makes it possible to dispense with conventional packaging. Indeed, the packaging of a solid composition according to the invention may be limited to a simple packaging in a paper.

Advantageously, a solid composition according to the invention does not consist of a fiber lattice.

Preferably, a composition according to the invention does not comprise any surfactant.

Preferably, a solid composition according to the invention does not comprise polyvinyl alcohol (PVA or PVOH), polyacrylate, copolymer of acrylic acid and methacrylic acid, polyvinylidene fluoride (PVDF), polymethyl methacrylate (PMMA), polyvinylpyrrolidone, polyalkylene oxide, polyacrylonitrile (PES or PAN), polymethacrylate of polyethylene terephthalate, polyethylene, polypropylene, nylon, hyaluronic acid, starch and derivatives thereof, pullulan, gelatin, cellulose (e.g. rayon), hydroxypropyl methylcellulose, methylcellulose, carboxymethyl cellulose, cotton, and one of the mixtures thereof.

A composition according to the invention does not comprise (or does not rest on) a water-soluble solid support, e.g. such as a hydrogel, more particularly based on starch, where appropriate destructured, and/or poloxamer 407.

### Manufacturing Method

A composition according to the invention is obtained by any method known to a person skilled in the art, suitable for the manufacture of a solid composition, provided the adaptations needed to ensure the formation of the cavities and thus to satisfy the required criterion of porosity.

Preferably, the method of manufacturing a solid composition according to the invention does not comprise a step of aeration/injection of a gas, e.g. air, where appropriate followed by a step of forming and then drying.

### Method No. 1

According to a first variant, a method of manufacturing a composition according to the invention comprises at least the steps consisting in:

(i) having available a composition in liquid or semi-liquid form;

(ii) casting the composition in a mold and drying until a solid composition is obtained,

(iii) cutting the solid composition into the desired shape, and

(iv) forming cavities to obtain a solid composition according to the invention.

Step (i) is advantageously carried out under hot conditions.

Advantageously, steps (iii) and (iv) are simultaneous.

Advantageously, steps (ii), (iii) and (iv) are simultaneous, in which case the mold has a shape suitable for ensuring the formation of the cavity (ies).

Step (iv) is advantageously carried out by means of a clay cutter.

Method No. 1 is advantageous in that the waste obtained at the end of step (iv) can be recovered during a subsequent manufacture. Indeed, the off-cuts can be heated again and added again in a subsequent method No. 1 at step (i) and/or (ii).

### Method No. 2

According to a second variant, a method of manufacturing a composition according to the invention is an extrusion method.

Such a manufacturing method according to the invention comprises at least the steps consisting of:

(a) having a composition in liquid or semi-liquid form;

(b) passing the composition of step (a) through an extrusion device equipped with a die (or extrusion grating) with a shape suitable for forming a solid bar, and

(c) cutting the bar obtained in step (b) to obtain a solid composition according to the invention.

For obvious reasons, step (b) is carried out at a temperature suitable for obtaining a composition with sufficient viscosity and apt to keep the shape given by the die.

For obvious reasons, the shape of the die of the extrusion device is also suitable for the formation of the cavities.

Advantageously, step (b) is carried out at a temperature lower than the ambient temperature, and e.g. at a temperature lower than or equal to 15° C., or even lower than or equal to 10° C.

In step (b), the extrusion device comprises a die having a shape suitable for forming at the solid bar the cavity (ies) and the desired level of porosity.

As an illustration, an extrusion method according to the invention comprises a die such as the die illustrated in FIG. 4.

Step (c) can be carried out using a knife or a wire, such as e.g. a butter cutting wire.

Preferably, steps (b) and (c) are simultaneous.

Preferably, the method of manufacturing a composition according to the invention is the extrusion method No. 2 described hereinabove.

According to a particular embodiment, applicable to methods No. 1 and No. 2 described above, step (i)/(a) consists in having a fluid composition which may comprise at least one solvent apt to evaporate, completely or partially, during step (ii)/(b), or even in addition during step (iii) and/or (iv)/(c).

As solvent, mention may be made e.g., of an apolar or slightly polar organic solvent, e.g. cyclohexane, THF (tetrahydrofuran), and a mixture thereof.

In the above methods No. 1 and No. 2, when the composition comprises at least one fatty phase comprising at least one lipophilic gelling agent, step (i)/(a) may require heating said composition to a temperature higher than the highest melting point of the lipophilic gelling agents present in said composition.

According to a particular embodiment, the extrusion method No. 2 can be carried out by a 3D printing technique.

### Use

A composition according to the invention is intended for make-up of a keratin material, more particularly of the skin and/or lips.

More particularly, a composition according to the invention may be e.g. a mascara, a complexion product, such as a foundation, an eyeliner, an illuminator/highlighter, a concealer, a dark circle concealer, a spot concealer, an eyeshadow or a blush, a lip product such as a lipstick, a plumper, a blush or a lip gloss, a conditioner, a khol or a self-tanner.

Advantageously, a solid composition according to the invention is not in the form of a mask.

According to a first embodiment, more particularly when a composition according to the invention is of the single-dose type, a solid composition according to the invention is intended for direct contact with a keratin material.

According to a second embodiment, a solid composition according to the invention is intended to be brought into contact with an aqueous phase, more particularly with water, before, simultaneously and/or after the application thereof to the keratin material to be treated. When a composition according to the invention is not of the single-dose type, the bringing in contact with water is carried out during the first use only, and presupposes a packaging of the “solid composition/water” mixture in a suitable container. For obvious reasons, thereof requires a solid composition of suitable formula and a sufficient quantity of water to ensure the transition from a solid composition to a liquid composition, e.g. in gel form. Thereof also requires the presence in the solid composition of at least one preservative in order to guarantee good preservation over time of the liquid composition thereby formed.

Finally, the present invention also relates to a non-therapeutic cosmetic treatment method for make-up of a keratin material, more particularly of the skin and/or the lips, comprising at least one step of applying to said keratin material of at least one composition as defined hereinabove.

According to a particular embodiment, the present invention relates to a product comprising, or deriving from the bringing into contact between, at least one first solid composition as described hereinabove and at least one second liquid composition.

### First Solid Composition

The first composition, as defined by the present invention, may equally well be referred to as “first solid composition” or “solid composition”, and corresponds to a solid composition as described hereinabove.

### Second Liquid Composition

The second composition, as defined by the present invention, may equally well be referred to as “second liquid composition” or “liquid composition”.

According to a first variant, the second composition is aqueous.

According to a second variant, the second composition is anhydrous.

According to a third variant, the second composition is an emulsion. An emulsion may be a direct (i.e. oil-in-water), inverse (i.e. water-in-oil) or multiple emulsion, more particularly a double emulsion (i.e. water-in-oil-in-water, oil-in-water-in-oil or oil-in-oil-in-water).

According to a particular embodiment, the second composition is a stable emulsion, preferably direct, highly concentrated in dispersed phase, and in particular is an HIPE (i.e. “High Internal Phase Emulsion”) or an apollonian emulsion.

A second composition according to the invention is not in solid form at ambient temperature and at ambient pressure, i.e. same is apt to flow under its own weight thereof.

A second composition according to the invention is in a liquid form, preferably in the form of a gel.

The viscosity of a second composition or of at least the phase thereof when in the form of an emulsion, can vary significantly, which makes it possible to obtain varied textures. The viscosity is measured at ambient temperature and at ambient pressure according to the method described in WO2017046305.

For obvious reasons, the viscosity of the second composition is adjusted to make possible an easy withdrawal/administration of the product according to the invention.

According to one embodiment, the second composition advantageously has a viscosity comprised from 5,000 MPa.s to 500,000 MPa.s, preferentially from 10,000 MPa.s to 300,000 MPa.s, better still from 25,000 MPa.s to 100,000 MPa.s, and more particularly from 50000 MPa.s to 75,000 MPa.s, as measured at 25° C., according to the method described hereinabove.

Preferably, the second composition may reduce the solidity of the first composition. More particularly, the second composition may advantageously comprise at least one starting material apt to reduce the solidity of the first composition. Such embodiment is advantageous in that same makes it possible to change the solid texture of the first composition toward a softer texture. One can thereby speak of a “transformative effect”.

A product according to the invention may advantageously comprise a “first composition(s)/second composition(s)” weight ratio comprised between 0.01 and 10, preferably between 0.1 and 5 and better between 0.5 and 1.

According to a particular embodiment, the first composition may comprise high contents of pigment(s) and/or of biological/cosmetic active agent(s) as described hereinbelow. Thereby, the first composition can be considered to be a “booster”.

Given the foregoing, a product according to the invention is an extemporaneous product, i.e. when the product is first used, it is the user who will bring the first composition into contact with the second composition.

Thereby, the invention relates to a kit comprising:
at least one first composition as described hereinabove;
at least one second composition as described hereinabove; and
optionally, at least one container.

A container may be referred to, without distinction, as “container”, “packaging” or “primary packaging”.

The invention further relates to the use of the kit described hereinabove, for obtaining a product according to the invention.

Preferably, a kit according to the invention can further comprise at least one means of application/administration. In the case where the product is intended for cosmetic use, a kit according to the invention may also comprise at least one means of application to a keratin material, e.g. chosen from the group formed by spatulas, brushes, feathers or sponges.

According to a particular variant, the kit may comprise a plurality of first compositions, identical or different, and/or a plurality of second compositions, identical or different, and/or at least one means of application.

Thereby, the invention further relates to a kit comprising a plurality of first compositions in the form of single doses comprising different cosmetic/biological active agents and/or different contents of cosmetic/biological active agent(s).

Thereby, such a kit according to the invention advantageously makes it easy for the user to adapt to the desired effect.

In the case of a cosmetic product, such particular embodiment allows the user to decide on the desired cosmetic effect, in particular according to his/her state of mind (mood), his/her style of clothing and/or the time of day.

### BRIEF DESCRIPTION OF FIGURES

FIG. 1 shows a composition according to the invention, with a parallelepiped shape having a cavity of cylindrical shape. A is the side view and B is the top view.

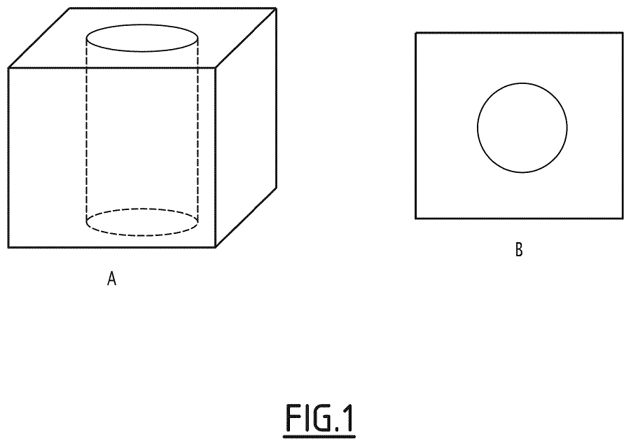

FIG. 2 represents an ovoid-shaped composition according to the invention with cavities (top view), representing the word “SOAP”.

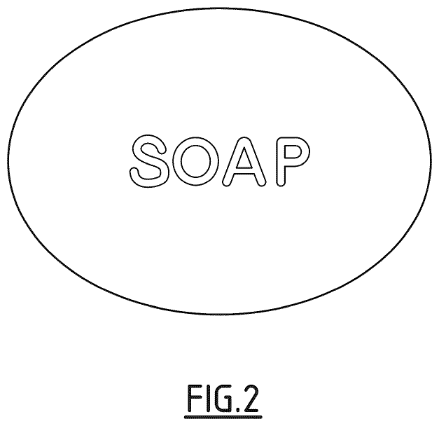

FIG. 3 shows a composition according to the invention with honeycomb cavities (top view).

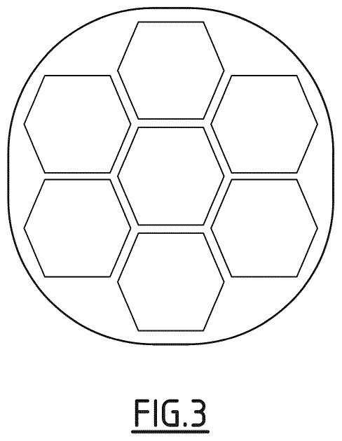

FIG. 4 shows a die used for the method of the invention.

FIG. 5 shows a comparative die.

FIG. 6 shows other examples of solid compositions according to the invention.

### EXAMPLES

### Example 1

### Comparative Study

### Step 1—Manufacturing:

In a container placed in a water bath (80° C.), all the ingredients listed in the table below are mixed until reaching homogenization.

| Name | % |
|---|---|
| Cocoa butter | QSP* |
| Bee wax (white) | 13.33 |
| Sweet almond oil | 16.33 |
| Calcium carbonate | 33.33 |
| Iron oxide | 8.34 |
| Total | 100 |

*Quantum satis

### Step 2—Extrusion:

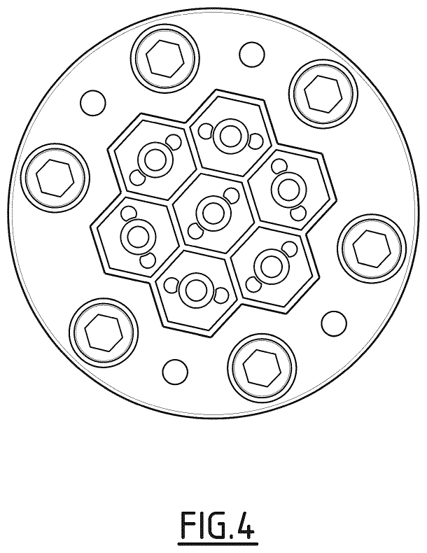

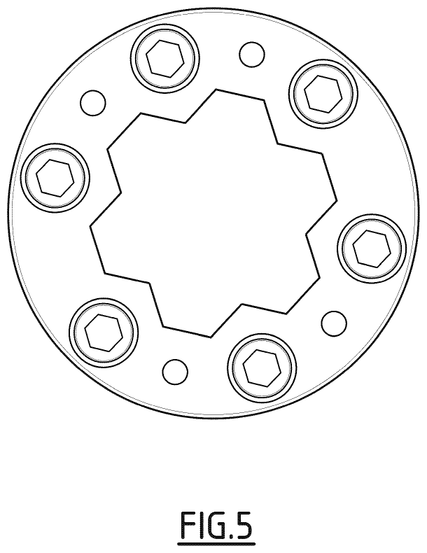

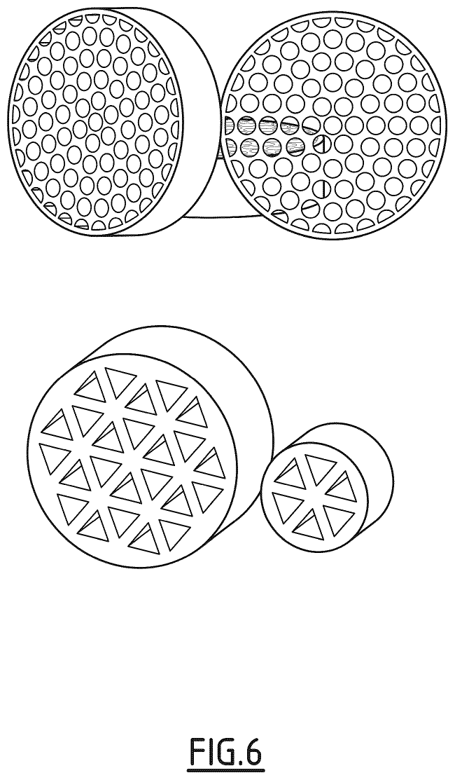

The mixture obtained in step 1 is injected into an extruder provided, in a first case, with a die according to the invention as illustrated in FIG. 4 and, in a second case, with a comparative die as illustrated in FIG. 5;
At the outlet of the die, the respectively extruded bars are cut so as to obtain the weights and volumes described in Table 1 below.
Single-dose solid make-up compositions are obtained in the form of a solid composition according to the invention with a honeycomb structure (test 1, like e.g. same described in FIG. 6) and a comparative solid composition with a general shape identical to test 1 but with no cavity (soaps 2 and 3), respectively.

| | Test 1 | Test 2 | Test 3 |
|---|---|---|---|
| | (invention) | (comparative) | (comparative) |
| Weight (mg) | 20 | 20 | 80 |
| Volume (in cm³) | 0.08 | 0.02 | 0.08 |
| Porosity (%) | >75 | 0 | 0 |

### Step 3—Application:

Step 3 is carried out on a panel of 18 women between the ages of 22 and 47 (hereinafter the “candidates”), 3 groups being then formed, each group being formed of 6 women (groups A, B and C).

For each group, the candidates apply the composition tested on the lips. Each of the groups A, B and C uses the tests 1, 2 and 3, but each time in different orders.

During the step 3, the candidates assess the performance of tests 1, 2 and 3 in terms of (i) gripping capacity, (ii) homogeneity of the coverage, (iii) ease of application and (iii) condition of the composition after use.

### Scoring Criteria:

| SCORING CRITERIA | 0 | 1 | 2 |
|---|---|---|---|
| Grip | Satisfactory | Average | Poor |
| Homogeneity of the | Satisfactory | Average | Poor |
| coverage | | | |
| Ease of application | Satisfactory | Average | Poor |
| Condition of the | Total | Some | General |
| composition after use | disappearance | residue | structure kept |

The means of the results obtained with the three groups A, B and C are presented in the following table, bearing in mind that the observed trends are identical between the three groups.

| | Test 1 | Test 2 | Test 3 |
|---|---|---|---|
| | (Invention) | (Comparative) | (Comparative) |
| Grip | 0 | 2 | 0 |
| Homogeneity of the | 0 | 1 | 1 |
| coverage | | | |
| Ease of application | 0 | 1 | 1 |
| Condition after use | 0 | 1 | 2 |

With comparative tests 2 and 3, it is observed that when the size is too small, the grip is poor and handling is difficult and when the product is too bulky, it is not possible to consume same completely during a single-dose use, which generates a lot of losses.

Furthermore, tests 2 and 3 have average properties in terms of coverage homogeneity and ease of application.

Test 1 according to the invention makes it possible to combine the advantages of comparative tests 2 and 3 without the drawbacks thereof.

A composition according to the invention, due to the presence of cavities in the contents considered, makes it possible to obtain solid make-up compositions combining an ability to generate a homogeneous coverage with less material while generating less loss and having a homogeneity and satisfactory ease of application, without prejudice to the grip-related properties.

A solid composition according to the invention thus consumes less raw materials and is thus both ecologically and economically advantageous.

A study similar to the study described hereinabove was also carried out with a solid composition such as a foundation, similar to same illustrated in example 2. Identical results were obtained.

### Example 2

### Solid Stick Foundation

| NAME | INCI | % | Phase |
|---|---|---|---|
| LABRAFAC CC | Caprylic/CapricTriglyceride | QSP* | A |
| CASTOR OIL | Ricinus Communis(Castor) SeedOil | 8.00 | A |
| ESTOGEL M | Castor Oil/IPDI Copolymer (and) | 30.00 | A |
| | Caprylic/CapricTriglyceride | | |
| ESTOGEL | Hydrogenated Castor Oil/Sebacic Acid | 8.00 | A |
| GREEN | Copolymer | | |
| LUSPLAN DD- | Dimer DilinoleylDimer Dilinoleate | 3.70 | A |
| DA7 | | | |
| COPHEROL | Tocopheryl Acetate | 0.50 | B |
| 1250 C | | | |
| SCENT COCO | Fragrance | 0.50 | B |
| BBF-35 | | | |
| ASL-1 TIO2 | CI77891, Aluminum hydroxide, Sodium | 15.40 | A |
| CR-50 | Lauroyl glutamate, Lysine, Magnesium | | |
| | chloride | | |
| ASL-1 YELLOW | CI 77492, Sodium Lauroyl glutamate, | 2.73 | A |
| LL-100P | Lysine, Magnesium chloride | | |
| ASL-1 RED R- | CI 77491, Sodium Lauroyl glutamate, | 0.70 | A |
| 516P | Lysine, Magnesium chloride | | |
| ASL-1 BLACK | CI 77499, Sodium Lauroyl glutamate, | 0.27 | A |
| BL-100P | Lysine, Magnesium choride | | |

*Quantum satis

The manufacturing method is the following:
Adding the ingredients of phase A into a container and heat the mixture with stirring at 100° C. until reaching homogenization;
Cooling to 80° C. and adding the ingredients of phase B with stirring until reaching homogenization;
Injecting the mixture obtained into an extruder equipped with a die according to the invention as illustrated in FIG. 4;
At the outlet of the die, cutting the extruded bars so as to obtain the desired shape and size; and
Packaging the solid composition obtained in a suitable packaging.

1. A solid cosmetic make-up composition of a keratin material, more particularly the skin and/or the lips, comprising at least one pigment and at least one cavity such that said composition comprises a porosity greater than or equal to 20% by volume relative to the total volume of said solid composition.

2. The solid composition according to claim 1, wherein the porosity is greater than or equal to 30% by volume relative to the total volume of said solid composition.

3. The solid composition according to claim 1, wherein the cavity (ies) is/are through.

4. The solid composition according to claim 1, said composition comprising one or a plurality of cavities, either identical or different.

5. The solid composition according to claim 1, wherein the cavity (ies) is/are round, oblong, ovoid, triangular, polygonal, honeycomb, or in the shape of a paving (or cutout).

6. The solid composition according to claim 1, wherein the ratio between the volume of the cavity (ies) and the total volume of the composition is comprised between 0.2 and 0.9.

7. The composition according to claim 1, wherein said composition comprises between 1% and 60% by weight of pigment(s) relative to the total weight of said composition.

8. The solid composition according to claim 1, wherein said composition comprises at least one lipophilic gelling agent, at least one oil, and a mixture thereof.

9. The solid composition according to claim 1, wherein said composition further comprises at least one aqueous phase dispersed in the solid composition.

10. The solid composition according to claim 1, wherein said composition is a single-dose composition.

11. A kit comprising:
at least a first composition comprising the solid composition according to claim 1;
at least one second liquid composition; and
optionally, at least one container.

12. A packaging comprising a plurality of the solid compositions according to claim 1, said compositions comprising different pigments and/or different contents of pigment(s) and therefore different shades.

13. The method of manufacturing the solid cosmetic composition according to claim 1, comprising at least the steps consisting of:
(i) having a composition in liquid or semi-liquid form;
(ii) casting the composition in a mold and drying until a solid composition is obtained,
(iii) cutting the solid composition into the desired shape, and
(iv) forming cavities for obtaining the solid composition.

14. The method of manufacturing the solid cosmetic composition according to claim 1, comprising at least the steps consisting of:
(a) having a composition in liquid or semi-liquid form;
(b) passing the composition of step (a) through an extrusion device equipped with a die with a shape suitable for forming a solid bar, and
(c) cutting the bar obtained during step (b) to obtain the solid composition.

15. A non-therapeutic method for cosmetic make-up treatment of a keratin material, comprising at least one step of applying to said keratin material at least the solid composition according to claim 1.
